(19) United States
(12) Patent Application Publication
(10) Pub. No.: US 2026/0198940 A1
(43) Pub. Date: Jul. 16, 2026

(54) MEASUREMENT DEVICE AND DRILL GUIDE

(71) Applicant: Arthrex, Inc., Naples, FL (US)

(72) Inventors: Patrick A. Smith, Naples, FL (US); Michael Morris, Naples, FL (US); Andrew Osika, Naples, FL (US); Matthew Johnson, Naples, FL (US)

(73) Assignee: Arthrex, Inc., Naples, FL (US)

(21) Appl. No.: 19/137,361

(22) PCT Filed: Dec. 21, 2023
(86) PCT No.: PCT/US2023/085360

§ 371 Date: Jun. 10, 2025

Related U.S. Application Data
(60) Provisional application No. 63/476,429, filed on Dec. 21, 2022.

(51) Int. Cl. A61B 17/17; A61B 17/00; A61B 17/04

(52) U.S. Cl. CPC A61B 17/1764; A61B 17/0401; A61B 2017/00738

(57) ABSTRACT

Medical instruments and methods that can be used in conjunction with surgical procedures that involve inserting one or more anchors into a joint. In an example, the disclosed instrument can be used in conjunction with surgical procedures in which the one or more anchors are to be inserted at an offset from a reference location in the joint. The disclosed instrument allows for inserting the anchor at a precise location from the reference location in the joint.

### PRIORITY

This application claims the benefit of U.S. Ser. No. 63/476,429, filed on Dec. 21, 2022, which is incorporated herein in its entirety by reference.

### BACKGROUND

The disclosure herein generally relates to medical instruments and, more particularly, to medical instruments and methods for anchor placement.

### SUMMARY

Existing methods of repairing a joint can involve inserting one or more anchors into the joint. However, current methods of repairing a joint by inserting one or more anchors into the joint have various drawbacks. For instance, it can be difficult to both efficiently and accurately insert the one or more anchors at the desired location.

Methods and systems in accordance with the present disclosure provide systems and methods for anchor placement in a joint. In example embodiments, the disclosed systems can be used during repair of a meniscal extrusion in which a meniscus is secured to a knee joint structure with one or more anchors.

In an example, a medical instrument is described. The medical instrument includes a handle at a proximal end. A shaft is connected to the handle. An offset drill guide is connected to the handle and located at a distal end. The offset drill guide includes one or more openings and a hook having a first portion that curves above a longitudinal axis of the shaft and a second portion that extends below the longitudinal axis of the shaft. The offset drill guide can further include a channel that extends from the one or more openings to the distal end, such that the hook is forked. The offset drill guide can further include one or more measurement lines between the one or more openings and the shaft.

In another example, a method is described. The method includes inserting a portion of a medical device into a knee joint space. The medical device includes a handle at a proximal end, a shaft connected to the handle, and an offset drill guide connected to the handle and located at a distal end. The offset drill guide includes one or more openings and a hook having a first portion that curves above a longitudinal axis of the shaft and a second portion that extends below the longitudinal axis of the shaft. A first portion of the offset drill guide is positioned beneath the meniscus and above a tibial plateau such that the hook extends over a tibial edge. A portion of a drill guide is inserted into the knee joint space. A distal tip of the drill guide is positioned in an opening of the one or more openings. A hole is drilled in the tibial plateau using a drill pin that extends through the drill guide. An anchor system is inserted through the drill guide and an anchor is mounted in the hole. The drill guide is removed from the knee joint space. The offset drill guide is removed from around the anchor system and from the knee joint space. The offset drill guide can include a channel that extends from the one or more openings to the distal end, such that the hook is forked and allows the anchor system to pass through during the removal of the offset drill guide.

### BRIEF DESCRIPTION OF THE DRAWINGS

For a better understanding of the examples described in this application, reference should be made to the Detailed Description below, in conjunction with the following drawings in which like reference numerals refer to corresponding parts throughout the figures.

FIG. 1A is a side view of a medical instrument, according to an example of the present disclosure;

FIG. 1B is a top view of an example of an offset drill guide, according to an example of the present disclosure;

FIG. 1C is a top view of another example of an offset drill guide, according to an example of the present disclosure;

FIG. 1D is a top view of another example of an offset drill guide, according to an example of the present disclosure; note that the hook is not depicted in FIG. 1B-1D;

FIG. 2A is a perspective view of a knee, according to an example of the present disclosure;

FIG. 2B is a perspective view of knee joint space illustrating the offset drill guide inserted through a contralateral portal, according to an example of the present disclosure;

FIG. 2C is a perspective view showing the offset drill guide lifting a meniscus, according to an example of the present disclosure;

FIG. 2D is a perspective view showing the offset drill guide lifting the meniscus, according to an example of the present disclosure;

FIG. 2E is a perspective view illustrating the insertion of a drill guide into one or more openings of the offset drill guide, according to an example of the present disclosure;

FIG. 2F is a perspective view illustrating the insertion of a drill pin through the drill guide, according to an example of the present disclosure;

FIG. 2G is a perspective view illustrating the insertion of an anchor system through the drill guide, according to an example of the present disclosure;

FIG. 2H is a perspective view illustrating the removal of the drill guide after placement of the anchor system, according to an example of the present disclosure; and

FIG. 2I is a top view of the joint space after removal of the medical instrument, according to an example of the present disclosure.

### DETAILED DESCRIPTION

The present disclosure will now be described more fully hereinafter with reference to the accompanying drawings, in which some, but not all of the disclosed examples are shown. Indeed, several different examples are described and should not be construed as limited to the examples set forth herein.

The present disclosure includes systems, methods, and medical instruments for anchor placement in a joint. In some examples, the disclosed systems, methods, and medical instruments can be used during repair of a meniscal extrusion, and resulting disruptions of tibiofemoral contact mechanics, in which the meniscus is secured to a knee joint structure with one or more anchors.

The disclosed medical instruments can be used in conjunction with surgical procedures that involve inserting one or more anchors into a joint. In an example, the disclosed instrument can be used in conjunction with surgical procedures in which the anchor(s) is to be inserted at an offset from a reference location in the joint. The disclosed instrument allows for inserting the anchor at a precise location from the reference location in the joint.

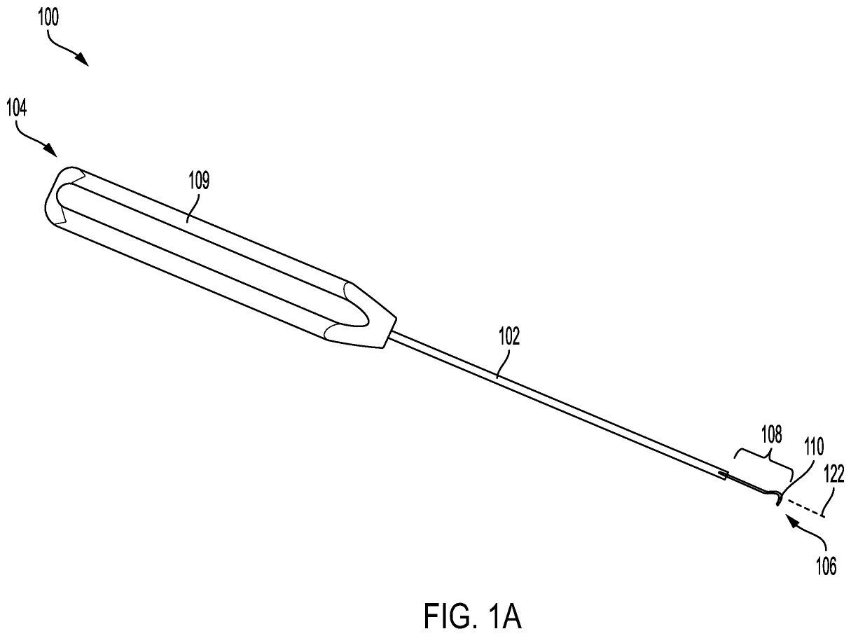

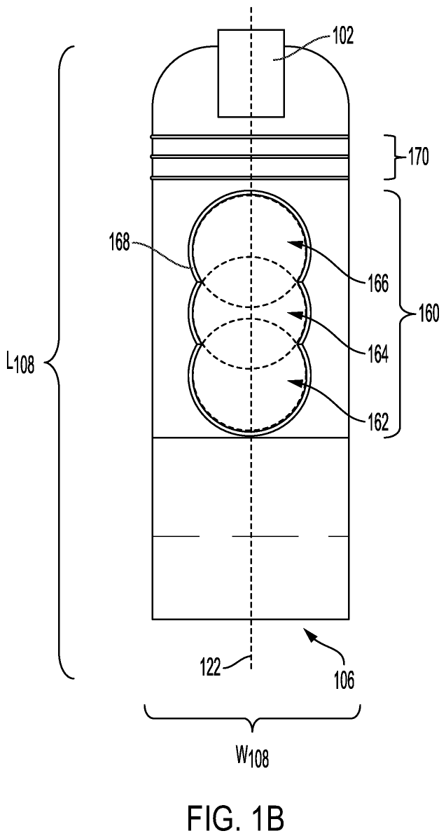

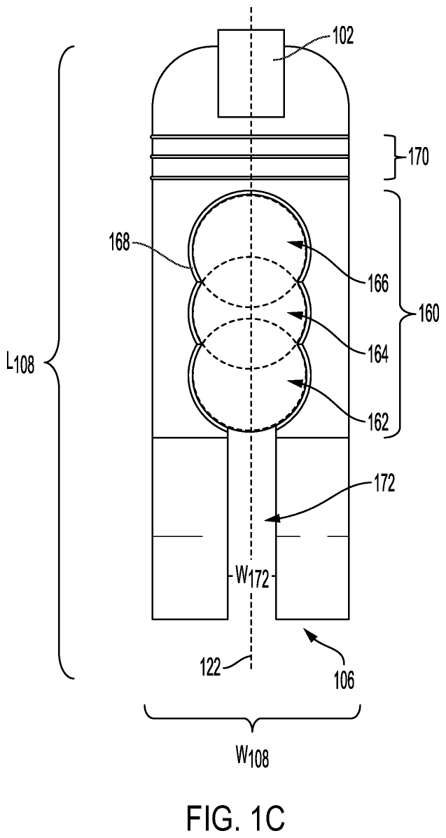

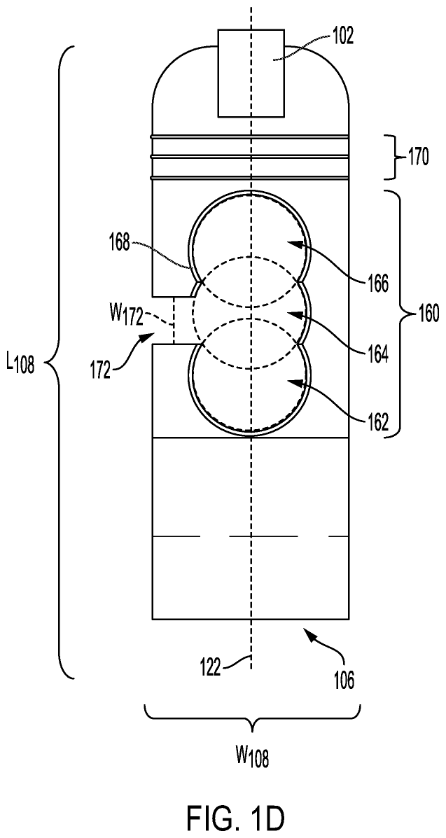

Referring now to FIGS. 1A-1C, an example medical instrument 100 for anchor placement is illustrated. FIG. 1A shows a side view of the medical instrument 100. The medical instrument 100 has a proximal end 104 and a distal end 106. The medical instrument 100 includes a handle 109 connected to a shaft 102. The shaft is connected to an offset drill guide 108. The offset drill guide 108 extends a predetermined length from the distal end 106 of the medical instrument 100 along a longitudinal axis 122 of the shaft 102.

Any suitable materials can be used for these components of the medical instrument 100, including but not limited to metal, plastic, and/or rubber. In an example, one or more of the shaft 102 and the offset drill guide 108 can be made of a medical-compliant metal, such as surgical-grade stainless steel. Other materials, such as nitinol, are possible as well. In the example shown, the medical handle 109 is located at the proximal end 104 of a medical instrument 100. The handle 109 can provide an ergonomic grip to allow a surgeon to hold and maneuver the medical instrument 100 as needed during a surgical procedure.

The shaft 102 can be solid or a hollow tube and can have any number of configurations (e.g., slotted, bar stock, flexible pattern/laser cut, etc.). In an example, the shaft 102 is circular or substantially circular in cross-section. In other examples, the shaft 102 comprises a polygon shape, such as a triangle, square, pentagon, hexagon, heptagon, octagon, nonagon, or decagon. Other shapes are possible as well. In an example, the shaft 102 can include a bend for anatomic placement. The shaft 102 can be pre-bent or could be a bendable shaft. Any type of bend is possible. For example, the shaft 102 may have a left bend or a right bend (when looking down on the longitudinal axis 122) in order to accommodate placement within a left or a right knee.

The shaft 102 can include any suitable length, outer diameter, and inner diameter if hollow. In an example, the length of the shaft is about 145 mm to about 215 mm (e.g., about 145, 155, 160, 165, 175, 180, 185, 190, 195, 200, 205, 210, or 215 mm (or any range from about 145 to about 215 mm)). In an example, the outer diameter of the shaft is about 2.6 mm to about 5.6 mm (e.g., about 2.6, 2.8, 3.0, 3.5, 4.0, 4.5, 5.0, 5.5 or 5.6 mm (or any range from about 2.6 mm to about 5.6 mm)). In an example in which the shaft 102 is hollow, the inner diameter is about 1.6 mm to about 4.1 mm (e.g., about 1.6, 2.0, 2.5, 3.0, 3.5, 4.0, and 4.1 mm (or any range from about 1.6 to 4.1 mm)). The outer and inner diameter of the shaft 102 are selected so that shaft 102 is suitable for use in the body.

The offset drill guide 108 is connected to the shaft 102. In an example, the offset drill guide 108 and the shaft 102 are a single piece. In another example, the offset drill guide 108 is connected to the shaft 102 via a fusion, welding, crimping, or soldering process. The offset drill guide 108 is solid and substantially flat (other than the hook region). In an example, the offset drill guide 108 has a thickness of about 0.5 mm to about 1 mm, although other dimensions are contemplated. At the distal end 106, the offset drill guide 108 includes a hook 110, A first portion of the hook 110 curves or extends above the longitudinal axis 122 of the shaft 102. A second portion of the hook 110 curves or extends below the longitudinal axis 122 of the shaft 102. In an aspect, a first portion of the hook 110 curves or extends above the longitudinal axis by about 2.0, 1.9, 1.8, 1.7, 1.6, 1.5, 1.4, 1.3, 1.2, 1.1, 1.0, 0.9, 0.8, 0.7, 0.6, 0.5, 0.5, 0.3, 0.2, 0.1 mm or less. In an aspect, a second portion of the hook 110 curves or extends below the longitudinal axis by about 2.0, 1.9, 1.8, 1.7, 1.6, 1.5, 1.4, 1.3, 1.2, 1.1, 1.0, 0.9, 0.8, 0.7, 0.6, 0.5, 0.5, 0.3, 0.2, 0.1 mm or less.

FIG. 1B shows a top view of an example of the offset drill guide 108. In an example, the offset drill guide 108 has a length L₁₀₈ of about 0.75 in and a width of W₁₀₈ of about 0.25 in, although other dimensions are contemplated (e.g., a length of about 1.5, 1.0, 0.75, 0.5, 0.25 in or a width of about 0.5, 0.4, 0.3, 0.25, 0.2, or 0.1 in). The offset drill guide 108 includes one or more openings 160 (e.g., 1, 2, 3, 4, 5, or more). The one or more openings 160 can be any shape and size and any size to receive a drill guide (e.g., a distal end of a drill guide). For example, the one or more openings 160 can be circular or substantially circular. In another example, the one or more openings 160 can have a polygon shape, such as a triangle, square, pentagon, hexagon, heptagon, octagon, nonagon, or decagon. Other shapes are possible as well.

In an example, the one or more openings 160 are centered on the longitudinal axis 122 of the shaft 102. In another example, one or more of the one or more openings 160 are offset from the longitudinal axis 122 of the shaft 102 (e.g., offset by 0.01, 0.05, 0.1, 0.5, 1.0, 1.5, 1.7, 2.0 mm or more). The one or more openings 160 can include individual openings that are separated by a portion of the offset drill guide 108 or they can be concentric openings that overlap. The one or more openings 160 can be located any desired distance from the distal end 106. For example, the first opening 162 can be located about 4 mm, about 6 mm, or about 8 mm from the distal end 106. The one or more openings 160 can have a bevel 168 to assist in the placement of the drill guide.

In the example shown in FIG. 1B, the one or more openings 160 are concentric circles include a first opening 162, a second opening 164, and a third opening 166 although any number of openings is contemplated. The first opening 162, second opening 164, and third opening 166 can have one or more diameters. For example, the first opening 162, second opening 164, and third opening 166 can have diameter of about 1.6 mm to about 5.6 mm (e.g., about 1.6, 1.8, 2.0, 2.3, 2.5, 2.6, 2.8, 3.0, 3.5, 4.0, 4.1, 4.5, 5.0, 5.5 or 5.6 mm (or any range from about 1.6 mm to about 5.6 mm)). The first opening 162, second opening 164, and third opening 166 can have the same diameter, different diameters, or a mixture of diameters.

The offset drill guide 108 can include one or more measurement lines 170. The one or more measurement lines 170 can be etched into the offset drill guide 108 (e.g., by mechanical means or via a laser etching process). The one or more of the one or more openings 160 and the one or more measurement lines 170 can be used to readily visualize and determine an amount of meniscal extrusion.

FIG. 1C shows a top view of another example of the offset drill guide 108. As shown in FIG. 1C, the offset drill guide 108 can include a channel 172. In an example, the channel 172 extends from the first opening 162 to the distal end 106. The channel 172 facilitates the removal of the offset drill guide 108 from around any sutures and/or anchors. In an example, the channel 172 has a width W₁₇₂ of about 0.5 mm to about 7 mm (e.g., 0.1 , 0.3, 0.5, 0.75, 1.0, 2.0, 3.0, 4.0, 5.0, 6.0, 7.0, 8.0 9.0 mm or more), although other dimensions are contemplated.

FIG. 1D shows a top view of another example of the offset drill guide 108. As shown in FIG. 1D, the channel 172 extends laterally from the second opening 164 to a side of the offset drill guide 108 to facilitate the removal of the offset drill guide 108 from around any sutures and/or anchors. It should be noted that the channel 172 can extend laterally from any of the one or more openings 160 and can be located on either side of the offset drill guide 108.

The medical instrument 100 can be used in conjunction with surgical procedures that involve inserting one or more anchors into a joint, and is particularly suitable for surgical procedures in which the anchor(s) is to be inserted at an offset from a reference location in the joint that can be visualized (e.g., visualized with an arthroscope or other suitable device).

In accordance with examples, methods for anchor placement are provided. As indicated above, within examples, the medical instrument 100 can be used in surgical procedures in which a drill guide is needed to be positing at a determined distance to guide a drill pin for drilling a hole for an anchor. Typically, a surgeon would have to visually estimate the desired location at which to place the drill guide. In accordance with the present disclosure, the medical instrument 100 can be used to direct and locate a drill guide to place an anchor at a desired location (e.g., an offset position). Since the medical instrument 100 includes the offset drill guide 108, the medical instrument 100 allows for a more efficient and accurate anchor placement location than existing methods and devices.

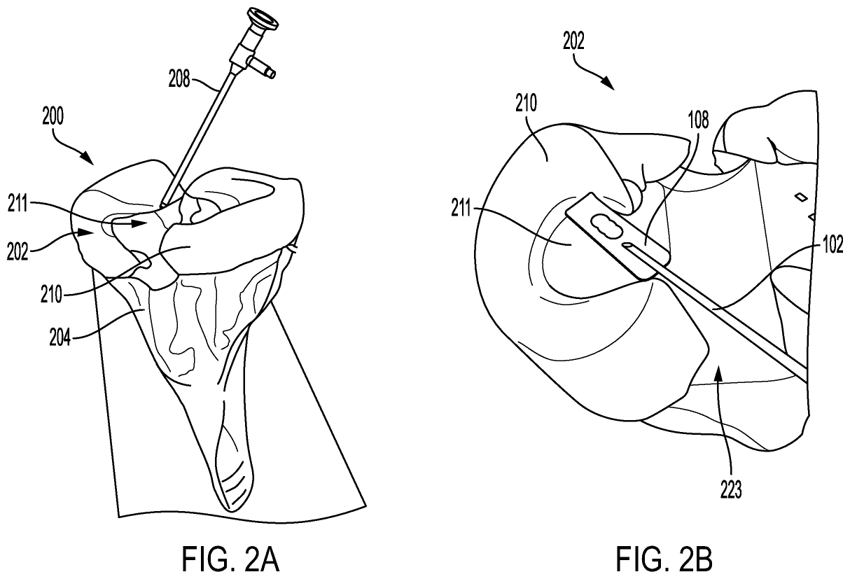

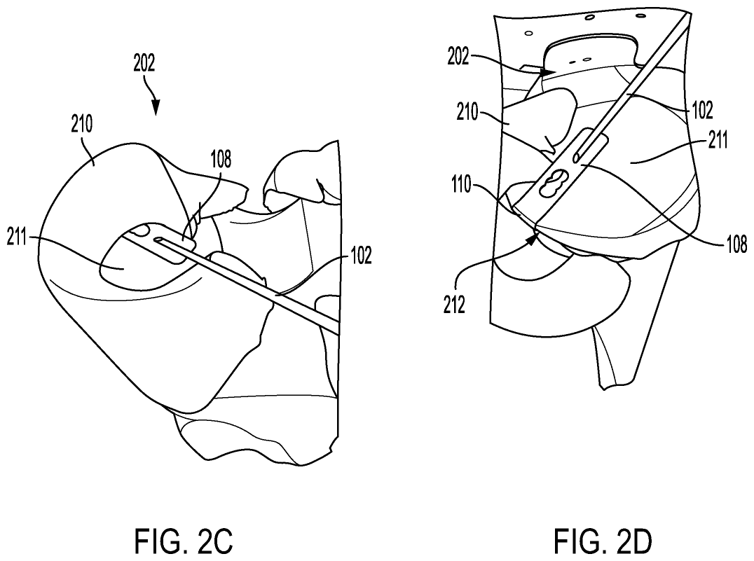

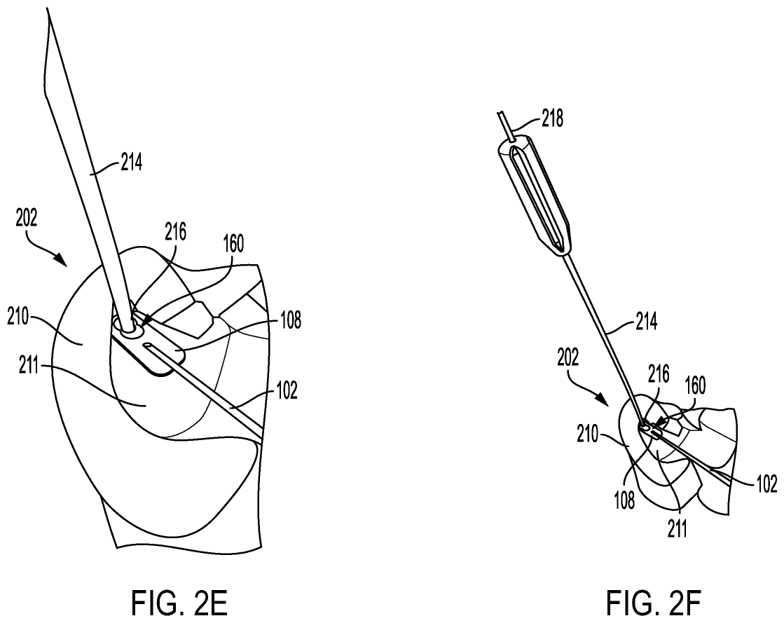

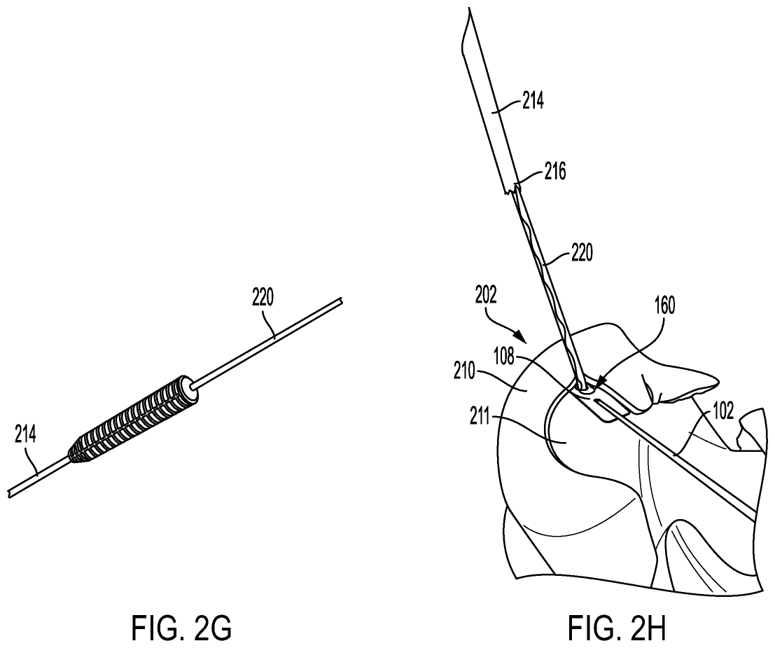

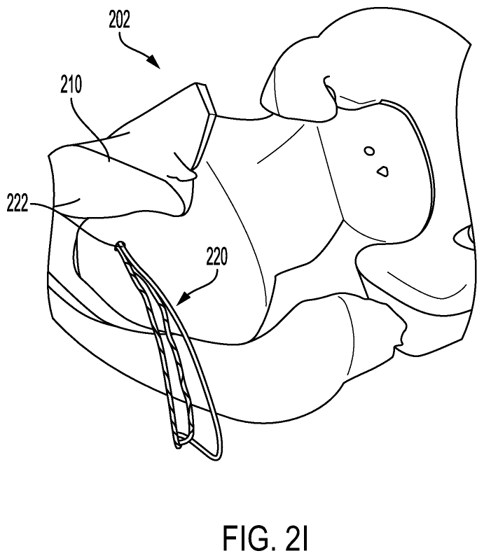

The offset drill guide 108 defines or identifies an anchor placement location that has a predetermined offset (e.g., a lateral distance) from a tibial edge. The example method can also involve advancing a drill guide through the one or more openings 160 and drilling a hole into a joint structure at the defined anchor placement location. The example method can also involve advancing an anchor through the offset drill guide 108 and placing the anchor in the hole. This example method is described in detail below with reference to FIGS. 2A-2I.

Referring now to FIG. 2A, a perspective view of a knee 200 upon which an example method is performed is shown. The knee 200 is illustrated as a synthetic knee model. However, in other examples, the knee 200 is a knee of a patient. In yet other examples, the knee 200 is a cadaveric knee. The knee 200 includes a knee joint space 202 and a knee joint structure 204 within a knee joint capsule. The example method involves inserting the medical instrument 100 into the knee joint space 202 and, in particular, through a knee capsule (not shown), beneath meniscus 210, and above tibial plateau 211. An arthroscope 208 is also inserted into the knee joint space 202. The arthroscope 208 can allow for visualization of the knee joint space 202, the meniscus 210, and the knee joint structure 204. The example method also involves visualizing the medical instrument 100 with arthroscope 208 to confirm that the offset drill guide 108 is located beneath meniscus 210 and above tibial plateau 211.

Referring now to FIG. 2B, a perspective view of the knee joint space 202 illustrating the offset drill guide 108 inserted through a contralateral portal 223 is shown. It should be noted that FIG. 2B show the offset drill guide 108 without the channel 172, but the methods described herein can be performed using the offset drill guide 108 with the channel 172.

Referring now to FIGS. 2C-2D, perspective views showing the offset drill guide 108 lifting the meniscus 210 and the hook 110 extending over a tibial edge 212. It should be noted that in FIG. 2D, the meniscus 210 has been moved for illustrative purposes to better show the hook 110 extending over the tibial edge 212.

Referring now to FIG. 2E, a perspective view illustrating the insertion of a drill guide 214 into the one or more openings 160 of the offset drill guide 108 is shown. The drill guide 214 can be inserted beneath the lifted meniscus 210 such that a distal tip 216 of the drill guide 214 is in contact with the tibial plateau 211. The drill guide 214 can be a straight or substantially straight drill guide or it can be a curved drill guide. The drill guide 214 can be rigid or flexible. In an example, the drill guide 214 is a Knotless FiberTak® drill guide.

In an example, the drill guide 214 has the same cross-sectional shape as one or more of the one or more openings 160. The distal tip 216 of the drill guide 214 can be positioned in any one of the one or more openings 160 based on a desired offset distance from the tibial edge 212. For example, the distal tip 216 of the drill guide 214 can be positioned in one or more of the first opening 162, the second opening 164, and the third opening 166.

Referring now to FIG. 2F, a perspective view illustrating the insertion of a drill pin 218 through the drill guide 214 is shown. Once the distal tip 216 of the drill guide 214 is positioned in the desired opening of the one or more openings 160, the drill pin 218 is advanced through the length of the drill guide 214 until it extends through the distal end 216 and contacts the tibial plateau 211. A hole 222 is drilled into the tibial plateau 211 at the defined offset location at the distance from the tibial edge 212. The drill pin 218 can be rigid or flexible. In an example, the drill pin 218 is a flexible FiberTak® drill pin.

Referring now to FIG. 2G, a perspective view illustrating the insertion of an anchor system 220 through the drill guide 214 is shown. The anchor system 220 is advanced through the length of the drill guide 214 until it extends through the distal end 216 and into the hole 222 drilled into the tibial plateau 211. Any suitable anchor systems are possible. For instance, the anchor system 220 can be a suture anchor (knotless or knotted; e.g., a Suture Tak® anchor), Alternatively, the anchor system 220 can be a staple. Other anchors are possible as well.

In an example, the anchor system 220 is a knotless FiberTak® anchor system comprising an “all-suture” soft-tissue fixation device with an expandable push-in design that is constructed from a hollow braid of polyester. An ultrahigh molecular weight polyethylene (UHMWPE) suture construct is assembled through the hollow braid coupled with a nitinol passing wire. The soft anchor can be provided preloaded on a disposable inserter.

The knotless suture constructs and systems described herein are used in conjunction with any knotless fixation devices which are pre-loaded with a flexible strand forming a splice within or outside the body of the fixation device. The fixation devices can be any of swivel and/or screw-in suture anchors and/or push-in suture. The fixation devices can be also any anchors, implants or screws (such as interference screws or tenodesis screws) or any fixation element that allows attachment/fixation of the knotless suture construct to bone. The fixation devices/implants can have various sizes (various diameters and/or lengths) and can be formed of biocompatible materials such as PEEK, biocomposite materials, metals and/or metal alloys, or combination of such materials, among others. The fixation devices can be unitary or can be multiple-piece constructs.

The flexible strand can be a high-strength suture, such as an UHMWPE suture without a core as this material allows easy splicing. Alternatively, the high strength suture can be a FiberWire® suture. The FiberWire® suture is formed of an UHMWPE, sold under the tradenames Spectra (Honeywell) and Dyneema (DSM) fibers, braided with at least one other fiber, natural or synthetic, to form lengths of suture material. The knotless suture constructs can also include sutures that are spliced—at least in part—in a manner similar to an Arthrex ACL TightRope® construct.

The anchor assembly described herein advantageously minimizes suture handling and management. The use of knotless anchors (such as push-in or screw-in type anchors) also provides secure fixation of the suture construct—the secure suture construct results from the suture being pushed into a hole and held tightly by anchors. The suture assembly employed in conjunction with the knotless anchor can also allow for knotless tensioning of the tissue after the plurality of knotless anchors have been implanted.

Referring now to FIG. 2H, a perspective view illustrating the removal of the drill guide 214 after placement of the anchor system 220 is shown.

Referring now to FIG. 2I, a top view of the joint space 202 after removal of the medical instrument 100 is shown. It should be noted that the meniscus 210 has been moved for illustrative purposes to better show the anchor system 220 and the hole 222. In an example, the channel 172 of the offset drill guide 108 permits sutures (e.g., a flexible material and a strand) of the anchor system 220 to pass through it, facilitating the removal of the offset drill guide 108 without disturbing the anchor system 220 from its position. The anchor system 220 remains implanted within the hole 222 at the desired offset location.

A meniscus centralization technique can be completed using the anchor system 220. In an example, a meniscus centralization technique includes passing a flexible material attached to a strand of the anchor system 220 through the meniscus 210, which is to be fixated (or reattached) to the tibial plateau 211; positioning the flexible material on a surface (for example, top surface) of the meniscus; and pulling on a free end of the strand to reduce the perimeter of a spliced adjustable loop and to cause the flexible material to bunch up on the surface of the meniscus 210, and to allow the meniscus 210 to achieve the desired location relative to the tibial plateau 211 upon proper tensioning. In an example, the knotless FiberTak® system is used, which will reduce to the offset location.

A method includes inserting the one or more anchors at the desired anchor placement location and the desired anchor placement offset. The disclosed methods can beneficially improve the efficiency and/or accuracy of anchor placement during surgical procedures for repairing a joint.

The offset drill guide 108 and the drill guide 214 can be inserted into the joint space 202. In an example, the offset drill guide 108 is inserted into the joint space 202 via the contralateral portal.

The distal end 106 of the offset drill guide 108 can be used to lift the meniscus 210 and locate the edge of the tibial plateau 211. In an example, the hook 110 is used to hook over the tibial edge 212.

The distal tip 216 of the drill guide 214 can be positioned in one or more openings 160 of the offset drill guide 108.

The hole 222 can be drilled in the tibial plateau 211 using a drill pin 218 that extends through the drill guide 214.

The anchor system 220 can be inserted through the drill guide 214 and mounted in the hole 222.

Upon completion of the procedures, the drill guide 214 should be removed from the joint space 202.

The offset drill guide 108 is removed from around the anchor system 220. In an example, the offset drill guide 108 can be removed from around the anchor system 220 by allowing the anchor system 220 to pass through the channel 172 as the offset drill guide 108 is removed. The channel 172 of the offset drill guide 108 permits sutures (e.g., a flexible material and a strand) of the anchor system 220 to pass through it, facilitating the removal of the offset drill guide 108 without disturbing the anchor system 220 from its position. The offset drill guide 108 is then removed from the joint space 202.

Although the examples illustrated in FIGS. 2A-2I include inserting one anchor system 220, more or fewer anchor systems 220 are possible. Furthermore, the anchor system 220 can be inserted through one or more of the medial knee capsule and the lateral capsule. Still further, the anchor system 220 can be into instated into one or more of the tibia and the femur.

Although the examples illustrated in FIGS. 2A-2I illustrate the joint space 202 as a knee joint, other joint spaces are possible as well. For instance, in other examples, the joint space is a shoulder joint, an elbow joint, or a wrist joint.

The disclosed methods and systems described herein beneficially provide improved methods and systems for anchor placement in a joint. The disclosed methods and systems allow for easily and accurately inserting the one or more anchors at the desired anchor placement location and the desired anchor placement position. Disclosed methods and systems beneficially can improve the efficiency and/or accuracy of anchor placement during surgical procedures for repairing a joint.

The term “joint space” refers to the space in a joint between two bones.

The term “joint capsule” refers to an envelope surrounding a synovial joint, where the joint capsule includes an outer fibrous layer or membrane and an inner synovial layer or membrane. On the inside of the joint capsule, articular cartilage covers the end surfaces of the bones that articulate within that joint. The joint capsule surrounds the bones joined by the synovial joint to provide strength and lubrication. The term “knee joint capsule” refers to an envelope that surrounds the knee joint and includes an outer fibrous layer or membrane and an inner synovial layer or membrane. The knee joint capsule surrounds the bones of the knee to provide strength and lubrication.

The term “knee joint structure” refers to the portions of the knee enveloped by and surrounding the knee joint capsule. In an example, the knee joint structure includes meniscus, the tibia, the femur, tibial periosteum, and femoral periosteum.

The term “tibial plateau” refers to the smooth bony surface of either the lateral condyle or the medial condyle of the tibia that articulates with the corresponding condylar surface of the femur.

From the foregoing, it will be appreciated that specific examples of the description have been described herein for purposes of illustration, but that various modifications can be made without deviating from the spirit and scope of the various examples of the description. Further, while various advantages associated with certain examples of the description have been described above in the context of those examples, other examples can also exhibit such advantages, and not all examples need to exhibit such advantages to fall within the scope of the description. Accordingly, the description is not limited, except as by the appended claims.

While the above description describes various examples of the description and the best mode contemplated, regardless how detailed the above text, the description can be practiced in many ways. Details of the system can vary considerably in its specific implementation, while still being encompassed by the present disclosure. As noted above, particular terminology used when describing certain features or aspects of the description should not be taken to imply that the terminology is being redefined herein to be restricted to any specific characteristics, features, or aspects of the description with which that terminology is associated. In general, the terms used in the following claims should not be construed to limit the description to the specific examples disclosed in the specification, unless the above Detailed Description section explicitly defines such terms. Accordingly, the actual scope of the description encompasses not only the disclosed examples, but also all equivalent ways of practicing or implementing the description under the claims.

The teachings of the description provided herein can be applied to other systems in addition to the system described above. The elements and acts of the various examples described above can be combined to provide further implementations of the description. Some alternative implementations of the description can include not only additional elements to those implementations noted above, but also can include fewer elements. Further any specific numbers noted herein are only examples: alternative implementations can employ differing values or ranges.

References throughout the foregoing description to features, advantages, or similar language do not imply that all of the features and advantages that can be realized with the present description should be or are in any single example of the description. Rather, language referring to the features and advantages is understood to mean that a specific feature, advantage, or characteristic described in connection with an example is included in at least one example of the present description. Thus, discussion of the features and advantages, and similar language, throughout this specification can, but do not necessarily, refer to the same example.

Furthermore, the described features, advantages, and characteristics of the present description can be combined in any suitable manner in one or more examples. One skilled in the relevant art will recognize that the present description can be practiced without one or more of the specific features or advantages of a particular example. In other instances, additional features and advantages can be recognized in certain examples that cannot be present in all examples of the present description.

The term “substantially” refers to a recited characteristic need not be achieved exactly, but that deviations or variations, including for example, tolerances, measurement error, measurement accuracy limitations and other factors known to persons having skill in the art, can occur in amounts that do not preclude the effect the characteristic was intended to provide.

The term “about” in association with a numerical value refers to a value that can vary by 5%. For example, a value of “about 100” means 95 to 105 (or any value between 95 and 105).

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Any of the terms “comprising,” “consisting essentially of,” and “consisting of” can be replaced with either of the other two terms, while retaining their ordinary meanings. As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number can also include the plural or singular number respectively. The word “or,” in reference to a list of two or more items, covers all of the following interpretations of the word: any of the items in the list, all of the items in the list, and any combination of the items in the list.

Any single term, single element, single phrase, group of terms, group of phrases, or group of elements described herein can each be specifically excluded from the claims.

Whenever a range is given in the specification, for example, a distance range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

Although certain aspects of the description are presented below in certain claim forms, the applicant contemplates the various aspects of the description in any number of claim forms. Accordingly, the applicant reserves the right to pursue additional claims after filing this application to pursue such additional claim forms, in either this application or in a continuing application.

What is claimed is:

1. A medical instrument comprising:
a handle at a proximal end;
a shaft connected to the handle; and
an offset drill guide connected to the handle and located at a distal end, the offset drill guide comprising one or more openings and a hook having a first portion that curves above a longitudinal axis of the shaft and a second portion that extends below the longitudinal axis of the shaft.

2. The medical instrument of claim 1, wherein the offset drill guide further comprises:
a channel that extends from the one or more openings to the distal end, such that the hook is forked.

3. The medical instrument of claim 2, wherein the channel has a width of about 1mm.

4. The medical instrument of claim 1, wherein the offset drill guide has a length of about 0.75 in and a width of about 0.25 in.

5. The medical instrument of claim 1, wherein the shaft is substantially circular in cross-section.

6. The medical instrument of claim 1, wherein the offset drill guide is substantially flat.

7. The medical instrument of claim 1, wherein the offset drill guide is one or more of a single piece with the shaft and connected to the shaft via a fusion, welding, crimping, or soldering process.

8. The medical instrument of claim 1, wherein the one or more openings comprise concentric circles having a diameter of about 1.6 mm to about 5.6 mm.

9. The medical instrument of claim 1, wherein the one or more openings are located one of about 4 mm, about 6 mm, or about 8 mm from the distal end.

10. The medical instrument of claim 1, further comprising one or more measurement lines on the offset drill guide between the one or more openings and the shaft.

11. A method comprising:
inserting portion of a medical device into a knee joint space, the medical device comprising:
a handle at a proximal end,
a shaft connected to the handle, and
an offset drill guide connected to the handle and located at a distal end, the offset drill guide comprising one or more openings and a hook having a first portion that curves above a longitudinal axis of the shaft and a second portion that extends below the longitudinal axis of the shaft;
positioning a first portion of the offset drill guide beneath a meniscus and above a tibial plateau such that the hook extends over a tibial edge;
inserting a portion of a drill guide into the knee joint space;
positioning a distal tip of the drill guide in an opening of the one or more openings;
drilling a hole in the tibial plateau using a drill pin that extends through the drill guide;
inserting an anchor system through the drill guide and mounting an anchor in the hole;
removing the drill guide from the knee joint space; and
removing the offset drill guide from around the anchor system and from the knee joint space.

12. The method of claim 11, wherein the offset drill guide further comprises:
a channel that extends from the one or more openings to the distal end, such that the hook is forked and allows the anchor system to pass through during the removal of the offset drill guide.

13. The method of claim 12, wherein the channel has a width of about 1mm.

14. The method of claim 11, wherein the offset drill guide has a length of about 0.75 in and a width of about 0.25 in.

15. The method of claim 11, wherein the shaft is substantially circular in cross-section.

16. The method of claim 11, wherein the offset drill guide is substantially flat.

17. The method of claim 11, wherein the offset drill guide is one or more of a single piece with the shaft and connected to the shaft via a fusion, welding, crimping, or soldering process.

18. The method of claim 11, wherein the one or more openings comprise concentric circles having a diameter of about 1.6 mm to about 5.6 mm.

19. The method of claim 11, wherein the one or more openings are located one of about 4 mm, about 6 mm, or about 8 mm from the distal end.

20. The method of claim 11, further comprising one or more measurement lines on the offset drill guide between the one or more openings and the shaft.
